(19) United States
(12) Patent Application Publication
(10) Pub. No.: US 2024/0374597 A1
(43) Pub. Date: Nov. 14, 2024

(54) METHODS AND COMPOSITIONS FOR TREATMENT OF KRAS MUTANT CANCER

(71) Applicant: BOARD OF REGENTS, THE UNIVERSITY OF TEXAS SYSTEM, Austin, TX (US)

(72) Inventors: John V. HEYMACH, Houston, TX (US); Jacqulyne P. ROBICHAUX, Houston, TX (US)

(73) Assignee: BOARD OF REGENTS, THE UNIVERSITY OF TEXAS SYSTEM, Austin, TX (US)

(21) Appl. No.: 18/293,144

(22) PCT Filed: Jul. 29, 2022
(86) PCT No.: PCT/US2022/074317

§ 371 Date: Jan. 29, 2024

Related U.S. Application Data
(60) Provisional application No. 63/227,237, filed on Jul. 29, 2021.

(51) Int. Cl. A61K 31/517; A61K 31/519; A61P 35/00

(52) U.S. Cl. CPC A61K 31/517; A61K 31/519; A61P 35/00

(57) ABSTRACT

The present disclosure provides methods and compositions for treatment of KRAS mutant cancer. Aspects of the disclosure are directed to methods for treatment of KRAS mutant cancer, including KRAS mutant non-small cell lung cancer, comprising administering a KRAS inhibitor (e.g., sotorasib, adagrasib) and poziotinib. Also disclosed are pharmaceutical compositions comprising a KRAS inhibitor (e.g., sotorasib, adagrasib) and poziotinib.

### CROSS REFERENCE TO RELATED APPLICATIONS

This application claims priority to U.S. Provisional Application Ser. No. 63/227,237, filed Jul. 29, 2021, which is incorporated by reference herein in its entirety.

### BACKGROUND

### I. Field of the Invention

Aspects of the invention relates to at least the fields of cancer biology and medicine.

### II. Background

Early reports studying novel KRAS^{G12C} inhibitors (e.g., sotorasib, adagrasib) have shown that within hours of KRAS^{G12C} inhibition, KRAS mutant cells upregulated HB-EGF, an EGFR and HER4 ligand, and HB-EGF upregulation corresponded with synthesis of active KRAS^{G12C} protein to reactivate cell growth. In a separate study, in pre-clinical models of KRAS mutant lung cancer, inhibition of EGFR resulted in transient attenuation of KRAS-mediated tumorigenesis. However, upregulation of other ErbB family members including ErbB2 (HER2), ErbB3 (HER3), and ErbB4 (HER4), restored downstream signaling abating anti-tumor effects. Thus, adaptation to KRAS^{G12C} inhibitors limits treatment efficacy.

There exists a need for methods and compositions for overcoming limited efficacy of KRAS inhibitors, including KRAS^{G12C} inhibitors, for improved treatment of patients with KRAS mutant cancer.

### SUMMARY

Aspects of the present disclosure address certain needs in the field of cancer medicine by providing improved methods and compositions for treatment of KRAS mutant cancer. Accordingly, provided herein, in some aspects, are methods of treating a subject for KRAS mutant cancer comprising administering to the subject a KRAS inhibitor and poziotinib. Also disclosed are pharmaceutical compositions comprising a KRAS inhibitor and poziotinib. In some embodiments, the KRAS inhibitor is a KRAS^{G12C} inhibitor, such as sotorasib or adagrasib.

Embodiments of the disclosure include methods for treating a subject for a KRAS mutant cancer, methods for treating a subject for a KRAS^{G12C} mutant cancer, methods for treating a subject for KRAS^{G12C} non-small cell lung cancer, methods for detecting a KRAS^{G12C} mutation, methods for diagnosing a subject with a KRAS mutant cancer, and pharmaceutical compositions comprising a KRAS inhibitor and poziotinib. Methods of the present disclosure can include at least 1, 2, 3, or more of the following steps: administering a KRAS inhibitor, administering a KRAS^{G12C} inhibitor, administering a HER2-4 selective inhibitor, administering poziotinib, administering a composition comprising a KRAS^{G12C} inhibitor and poziotinib, detecting a KRAS mutation in a subject, detecting a KRAS^{G12C} mutation in a subject, diagnosing a subject as having KRAS mutant cancer, and administering an additional cancer therapy. Any one or more of the preceding steps may be excluded from embodiments of the disclosure. Pharmaceutical compositions can include one or more of: a KRAS inhibitor, a KRAS^{G12C} inhibitor, sotorasib, a pharmaceutically acceptable sotorasib salt, adagrasib, a pharmaceutically acceptable adagrasib salt, poziotinib, a pharmaceutically acceptable poziotinib salt, and a pharmaceutically acceptable excipient. Any one or more of the preceding components may be excluded from embodiments of the disclosure.

Disclosed herein, in some embodiments, is a method of treating a subject for KRAS mutant cancer comprising administering to the subject an effective amount of (a) a KRAS inhibitor and (b) poziotinib. In some embodiments, the KRAS inhibitor and poziotinib are administered substantially simultaneously. In some embodiments, the KRAS inhibitor and poziotinib are administered sequentially. In some embodiments, the KRAS inhibitor is administered prior to administering poziotinib. In some embodiments, the KRAS inhibitor is administered subsequent to administering poziotinib. In some embodiments, the KRAS inhibitor is a KRAS^{G12C} inhibitor. In some embodiments, the KRAS mutant cancer is KRAS mutant non-small cell lung cancer. In some embodiments, the KRAS mutant cancer is KRAS mutant colorectal cancer. In some embodiments, the KRAS mutant cancer is KRAS mutant pancreatic cancer.

Also disclosed herein, in some embodiments, is a method of treating a subject for KRAS^{G12C} non-small cell lung cancer, the method comprising administering to the subject an effective amount of (a) a KRAS^{G12C} inhibitor; and (b) poziotinib. In some embodiments, the KRAS^{G12C} inhibitor and poziotinib are administered substantially simultaneously. In some embodiments, the KRAS^{G12C} inhibitor and poziotinib are administered sequentially. In some embodiments, the KRAS^{G12C} inhibitor is administered prior to administering poziotinib. In some embodiments, the KRAS^{G12C} inhibitor is administered subsequent to administering poziotinib.

In some embodiments, the method further comprises detecting a KRAS^{G12C} mutation in the subject. In some embodiments, the KRAS^{G12C} inhibitor is sotorasib (AMG 510). In some embodiments, the KRAS^{G12C} inhibitor is adagrasib (MRTX849). In some embodiments, the subject was previously treated with a cancer therapy. In some embodiments, the cancer therapy comprised chemotherapy. In some embodiments, the cancer therapy comprised a KRAS inhibitor. In some embodiments, the subject was determined to be resistant to the cancer therapy. In some embodiments, the subject was not previously treated with a KRAS inhibitor. In some embodiments, the poziotinib is administered at a dose of between 0.1 mg and 50 mg. In some embodiments, the poziotinib is administered at a dose of at least, at most, about, or exactly 0.1. 0.2. 0.3. 0.4. 0.5. 0.6. 0.7. 0.8. 0.9. 1.0. 1.1. 1.2. 1.3. 1.4. 1.5. 1.6. 1.7. 1.8. 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg, or any range or value derivable therein. In some embodiments, the poziotinib is administered at a dose of between 1 mg and 25 mg. In some embodiments, the poziotinib is administered at a dose of between 1 mg and 5 mg. In some embodiments, the poziotinib is administered orally. In some embodiments, the KRAS inhibitor and poziotinib are administered at least, at most, or exactly 1, 2, 3, 4, 5, 6, or 7 times per day for multiple days. In some embodiments, the KRAS inhibitor and poziotinib are administered at least, at most, or exactly 1, 2, 3, 4, 5, 6, 7, 8, 9, 10, 11, 12, 13, 14, 15, 16, 17, 18, 19, 20, or 21 times per week for multiple weeks. In some embodiments, the KRAS inhibitor and poziotinib are administered once per day for multiple days. In some embodiments, the KRAS inhibitor and poziotinib are administered twice per day for multiple days. In some embodiments, the method further comprises administering to the subject an additional cancer therapy. In some embodiments, the additional cancer therapy comprises chemotherapy, radiotherapy, immunotherapy, or a combination thereof.

Also disclosed herein, in some embodiments, is a pharmaceutical composition comprising (a) a KRAS inhibitor; (b) poziotinib; and (c) a pharmaceutically acceptable excipient. In some embodiments, the KRAS inhibitor is a KRAS^{G12C} inhibitor. In some embodiments, the KRAS^{G12C} inhibitor is sotorasib (AMG 510). In some embodiments, the KRAS^{G12C} inhibitor is adagrasib (MRTX849). In some embodiments, the poziotinib is at a dose of between 0.1 mg and 50 mg. In some embodiments, the poziotinib is at a dose of at least, at most, about, or exactly 0.1. 0.2. 0.3. 0.4. 0.5. 0.6. 0.7. 0.8. 0.9. 1.0. 1.1. 1.2. 1.3. 1.4. 1.5. 1.6. 1.7. 1.8. 1.9. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg, or any range or value derivable therein. In some embodiments, the poziotinib is at a dose of between 1 mg and 25 mg. In some embodiments, the poziotinib is at a dose of between 1 mg and 5 mg.

Further disclosed herein, in some embodiments, is a method of treating a subject for KRAS^{G12C} non-small cell lung cancer, the method comprising administering to the subject, twice daily for multiple days, an effective amount of (a) a KRAS^{G12C} inhibitor; and (b) poziotinib at a dose of between 1 mg and 5 mg. In some embodiments, the poziotinib is at a dose of at least, at most, about, or exactly 0.1. 0.2. 0.3. 0.4. 0.5. 0.6. 0.7. 0.8. 0.9. 1.0. 1.1. 1.2. 1.3. 1.4. 1.5. 1.6. 1.7. 1.8. 1.9, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg, or any range or value derivable therein.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. FIG. 1A shows western blot results from treatment of H23 cells for 4 hours with sotorasib (AMG 510) at the shown concentrations, or DMSO control. FIG. 1B shows sotorasib (AMG 510) IC₅₀ values in H358, H1378, H1792, and H2030 cells when combined with either 100 nM afatinib, 100 nM poziotinib, or DMSO control. FIG. 1C shows adagrasib (MRTX849) IC₅₀ values in H358, H1378, H1792, and H2030 cells when combined with either 100 nM afatinib, 100 nM poziotinib, or DMSO control.

FIG. 2 shows pEGFR and pHER2 levels in NSCLC cell lines bearing KRAS G12C mutations treated with sotorasib or adagrasib for 4 hours.

FIGS. 3A-3B. FIG. 3A shows phosphorylation of ERBB family members in HCC44, H2122, and H358 NSCLC cells (all harboring KRAS G12C mutations) treated with adagrasib for 72 hours. FIG. 3B shows phosphorylation of ERBB family members in HCC44, H2122, and H358 NSCLC cells (all harboring KRAS G12C mutations) treated with sotorasib for 72 hours.

FIGS. 4A-4B. FIG. 4A shows sensitivity of Ba/F3 cells expressing EGFR, EGFR/HER2, HER2/HER3, HER2/HER4, HER3/HER4, and HER4 to poziotinib. FIG. 4B shows sensitivity of Ba/F3 cells expressing EGFR, EGFR/HER2, HER2/HER3, HER2/HER4, HER3/HER4, and HER4 to afatinib.

FIG. 5 shows selectivity of poziotinib for various receptors.

FIGS. 6A-6B. FIG. 6A shows resistance of H358 cells to treatment with a combination of adagrasib and exogenous EGF or NRG1. FIG. 6B shows resistance of H358 cells to treatment with a combination of sotorasib and exogenous EGF or NRG1.

FIGS. 7A-7B. FIG. 7A shows a synergistic effect upon treatment of H23, HCC44, H2122, and H1792 cells (NSCLC harboring KRAS G12C mutations) with sotorasib alone or in combination with poziotinib. FIG. 7B shows a synergistic effect upon treatment of H23, HCC44, H2122, and H1792 cells (NSCLC harboring KRAS G12C mutations) with adagrasib alone or in combination with poziotinib.

FIGS. 8A-8B. FIG. 8A shows phosphorylation of ERBB family members in HCC44, H2122, and H358 NSCLC cells (all harboring KRAS G12C mutations) treated with sotorasib alone or in combination with afatinib or poziotinib. FIG. 8B shows phosphorylation of ERBB family members in HCC44, H2122, and H358 NSCLC cells (all harboring KRAS G12C mutations) treated with adagrasib alone or in combination with afatinib or poziotinib.

FIGS. 9A-9B. FIG. 9A shows the effect of sotorasib treatment alone or in combination with poziotinib (pozi) or afatinib (afat) on tumor volume in a PDX model of KRAS G12C mutant NSCLC. FIG. 9B shows the effect of sotorasib treatment alone or in combination with poziotinib (pozi) or afatinib (afat) on progression free survival in a PDX model of KRAS G12C mutant NSCLC.

### DETAILED DESCRIPTION

The present disclosure is based, at least in part, on the surprising discovery that administration of poziotinib, described herein to have selectivity for HER2-4 over EGFR, synergistically enhances the cytotoxicity of KRAS^{G12C} inhibitors against KRAS mutant tumor cells. Accordingly, aspects of the disclosure are directed to methods for treatment of a subject having KRAS mutant cancer comprising administration of a KRAS inhibitor (e.g., a KRAS^{G12C} inhibitor such as sotorasib or adagrasib) and poziotinib. Also disclosed are pharmaceutical compositions comprising a KRAS inhibitor, poziotinib, and one or more pharmaceutically acceptable excipients. Further aspects and embodiments of the present disclosure are described further herein.

### I. Therapeutic Methods

Aspects of the disclosure are directed to compositions comprising therapeutically effective amounts of one or more cancer therapeutics and administration of such compositions to a subject or patient in need thereof. In some embodiments, the one or more cancer therapeutics comprise a KRAS inhibitor and poziotinib.

The compositions of the disclosure may be used for in vivo, in vitro, or ex vivo administration. The route of administration of a composition may be, for example, intracutaneous, subcutaneous, intravenous, oral, local, topical, and intraperitoneal administrations.

The therapy provided herein may comprise administration of a combination of therapeutic agents, such as a KRAS inhibitor and poziotinib. The therapies may be administered in any suitable manner known in the art. For example, the KRAS inhibitor and poziotinib may be administered sequentially (at different times) or concurrently (at or at approximately the same time; also “substantially simultaneously”). In some embodiments, the KRAS inhibitor and poziotinib are administered in a separate composition. In some embodiments, the KRAS inhibitor and poziotinib are in the same composition.

In some embodiments, the KRAS inhibitor and poziotinib are administered substantially simultaneously. In some embodiments, the KRAS inhibitor and poziotinib are administered sequentially. In some embodiments, the KRAS inhibitor, poziotinib, and an additional cancer therapy are administered sequentially. In some embodiments, the KRAS inhibitor is administered before administering poziotinib. In some embodiments, the KRAS inhibitor is administered after administering poziotinib.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, a therapeutic agent of the disclosure (e.g., a KRAS inhibitor, poziotinib) is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician. Different therapeutics may be administered via the same route of administration or via different routes of administration.

The treatments may include various “unit doses.” Unit dose is defined as containing a predetermined-quantity of the therapeutic composition. The quantity to be administered, and the particular route and formulation, is within the skill of determination of those in the clinical arts. In the case of intravenous administration, a unit dose need not be administered as a single injection but may comprise continuous infusion over a set period of time. In some embodiments, a unit dose comprises a single administrable dose.

In some embodiments, the KRAS inhibitor is administered at a dose of between 1 mg/kg and 5000 mg/kg. In some embodiments, the KRAS inhibitor is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range or value derivable therein. In some embodiments, the KRAS inhibitor is administered at a dose of at least, at most, about, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 mg, or any range or value derivable therein.

In some embodiments, a single dose of poziotinib is administered. In some embodiments, multiple doses of poziotinib are administered. In some embodiments, poziotinib is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range or value derivable therein. In some embodiments, poziotinib is administered at a dose of between 0.1 mg/kg and 100 mg/kg.

In some embodiments, the poziotinib is administered at a dose of at least, at most, about, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, or 150 mg, or any range or value derivable therein. In some embodiments, poziotinib is administered at a dose of between 0.1 mg and 50 mg. In some embodiments, poziotinib is administered at a dose of between 1 mg and 25 mg. In some embodiments, poziotinib is administered at a dose of between 1 mg and 5 mg.

The quantity to be administered, both according to number of treatments and unit dose, depends on the treatment effect desired. An effective dose is understood to refer to an amount necessary to achieve a particular effect. Furthermore, such doses can be administered at multiple times during a day, and/or on multiple days, weeks, or months. In some embodiments, a composition of the disclosure (e.g., poziotinib and a KRAS inhibitor) is administered to a subject 1, 2, 3, 4, 5, 6, 7, 8, 9, or 10 times per day, or more, for multiple days. In some embodiments, poziotinib and a KRAS inhibitor are administered to a subject once per day for multiple days. In some embodiments, poziotinib and a KRAS inhibitor are administered to a subject twice per day for multiple days. In some embodiments, a composition of the disclosure (e.g., poziotinib and a KRAS inhibitor) is administered to a subject 1, 2, 3, 4, 5, 6, 7, 8, 9, 10, 11, 12, 13, 14, 15, 16, 17, 18, 19, 20, or 21 times per week, or more, for multiple weeks.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

In some embodiments, poziotinib is administered to a subject in an amount of between 0.1 mg and 50 mg, or any range or value derivable therein. Poziotinib, which may be administered in a hydrochloride salt form, may be administered orally, such as in a tablet. The poziotinib may be administered at a dose of between 1 and 25 mg, such as at a dose of 1, 2, 3, 4, 5, 6, 7, 8, 9, 10, 11, 12, 13, 14, 15, 16, 17, 18, 19, 20, 21, 22, 23, 24, or 25 mg, or any range or value derivable therein. The dosing may be twice daily, daily, every other day, every 3 days, or weekly. In some embodiments, the dosing is twice per day. The dosing may be on a continuous schedule, such as on 28 days cycles.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral administration and or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective.

### A. Cancer Therapy

In some embodiments, the method comprises administering a cancer therapy to the patient. The cancer therapy may be chosen based on expression level measurements, alone or in combination with a clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. In some embodiments, the cancer therapy is a KRAS inhibitor. In some embodiments, the cancer therapy is poziotinib. Any of these cancer therapies may also be excluded. Combinations of these therapies may also be administered. For example, as disclosed herein, a combination of a KRAS inhibitor and poziotinib is synergistically effective in treating KRAS mutant cancer; accordingly aspects of the disclosure are directed to a cancer therapy comprising a combination of a KRAS inhibitor (e.g., sotorasib or adagrasib) and poziotinib.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is non-small cell lung cancer. In some embodiments, the cancer is colorectal cancer. In some embodiments, the cancer is pancreatic cancer. In some embodiments, the cancer is KRAS mutant cancer. In some embodiments, the KRAS mutant cancer is KRAS G12C mutant cancer.

In some embodiments, the disclosed methods comprise treating a subject suffering from cancer (e.g., KRAS mutant cancer) by administering a therapeutically effective amount of a KRAS inhibitor and poziotinib. As used herein, the term “therapeutically effective amount” is synonymous with “effective amount.” “therapeutically effective dose,” and/or “effective dose,” and refers to an amount of an agent (or combination of agents) sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. For instance, in some embodiments, the effective amount refers to the amount of a KRAS inhibitor and poziotinib that, in combination, can treat or prevent cancer in a subject. The effective amount may vary depending on the organism or individual treated. The appropriate effective amount to be administered for a particular application of the disclosed methods can be determined by those skilled in the art, using the guidance provided herein. As used herein, the terms “treatment,” “treat,” or “treating” refers to intervention in an attempt to alter the natural course of the subject being treated, and may be performed either for prophylaxis or during the course of pathology of a disease or condition. Treatment may serve to accomplish one or more of various desired outcomes, including, for example, preventing occurrence or recurrence of disease, alleviation or reduction in severity of symptoms, and diminishment of any direct or indirect pathological consequences of the disease, preventing disease spread, lowering the rate of disease progression, amelioration or palliation of the disease state, and remission or improved prognosis.

### 1. KRAS Mutant Cancer

Aspects of the present disclosure are directed to methods for treatment of KRAS mutant cancer. As used herein, “KRAS mutant cancer” describes cancer harboring one or more KRAS mutations. Thus, a subject having KRAS mutant cancer describes a subject having cancer, where cancer cells from the subject have been identified as having a KRAS mutation. KRAS mutations include, for example, G12 mutations (e.g., G12A, G12C, G12D, G12R, G12V), G13 mutations (e.g., G13D), and Q61 mutations (e.g., Q61K, Q61L, Q61H). In some embodiments, a subject of the disclosure has KRAS G12C mutant non-small cell lung cancer (NSCLC). In some embodiments, a subject of the disclosure has KRAS G12C mutant colorectal cancer. In some embodiments, a subject of the disclosure has KRAS G12C mutant pancreatic cancer.

### B. KRAS Inhibitors

Aspects of the present disclosure comprise KRAS inhibitors and methods of use thereof. As used herein, a “KRAS inhibitor” describes any molecule capable of inhibiting activity of and/or reducing expression of a GTPase KRas (“KRAS” or “K-Ras”) protein. In some embodiments, a KRAS inhibitor is an oligonucleotide capable of reducing expression of a KRAS protein in a cell. In some embodiments, a KRAS inhibitor is an inhibitor of KRAS enzymatic activity. In some embodiments, a KRAS inhibitor is a molecule capable of inactivating KRAS by trapping KRAS in a GDP-bound state. In some embodiments, a KRAS protein targeted by a KRAS inhibitor of the disclosure is a mutant KRAS protein. Mutant KRAS proteins include, for example, KRAS proteins having a G12 mutation, such as G12A, G12C, G12D, G12R, or G12V. In some embodiments, a mutant KRAS protein is a KRAS protein having a G12C mutation (“KRAS^{G12C}”).

In some embodiments, a KRAS inhibitor of the disclosure is a KRAS^{G12C} inhibitor. As used herein, a “KRAS^{G12C} inhibitor” describes any molecule capable of inhibiting activity of and/or reducing expression of KRAS^{G12C}. In some embodiments, a KRAS^{G12C} inhibitor is a compound capable of trapping KRAS^{G12C} in a GDP-bound state, thus inhibiting enzymatic activity of the protein. In some embodiments, a KRAS^{G12C} inhibitor preferentially inhibits a KRAS^{G12C} protein relative to a wildtype KRAS protein. In some embodiments, a KRAS^{G12C} inhibitor does not inhibit a wildtype KRAS protein. Examples of KRAS^{G12C} inhibitors include, but are not limited to, ARS-1620, ARS-853, sotorasib (AMG 510), and adagrasib (MRTX849). In some embodiments, the KRAS^{G12C} inhibitor is 6-Fluoro-7-(2-fluoro-6-hydroxyphenyl)-(1M)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one, or sotorasib, having a structure represented by formula I, or a pharmaceutically acceptable salt thereof. In some embodiments, the KRAS^{G12C} inhibitor is {(2S)-4-[7-(8-chloronaphthalen-1-yl)-2-{[(2S)-1methylpyrrolidin-2-yl]methoxy}-5,6,7,8tetrahydropyrido[3,4-d]pyrimidin-4-yl]-1-(2-fluoroprop2-enoyl)piperazin-2-yl}acetonitrile, or adagrasib, having a structure represented by formula III, or a pharmaceutically acceptable salt thereof.

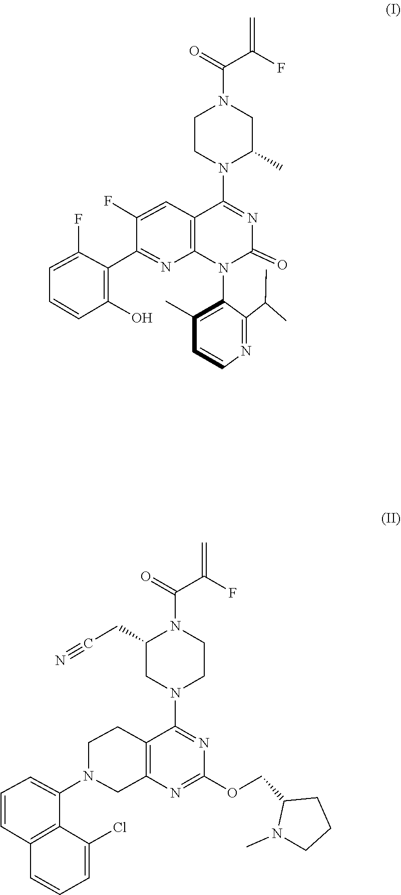

### C. Poziotinib

1-[4-[4-(3,4-dichloro-2-fluoroanilino)-7-methoxyquinazolin-6-yl]oxypiperidin-1-yl]prop-2-en-1-one, or poziotinib (also called “HM781-36” or “HM781-36B”), is a compound having a structure represented by formula III:

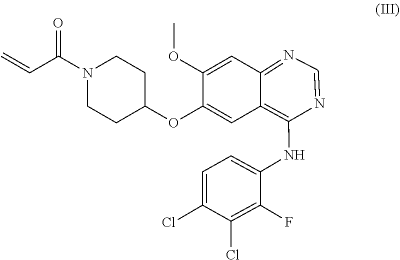 [SMILES: C=CC(=O)N1CCC(Oc2cc3c(Nc4ccc(Cl)c(Cl)c4F)ncnc3cc2OC)CC1]

Poziotinib is a pan-HER inhibitor, capable of inhibiting activity of ErbB1 (EGFR), ErbB2 (HER2), ErbB3 (HER3) and ErbB4 (HER4). Poziotinib is described in, for example, PCT Application Publication No. WO 2020/005932 and Cha M Y., et al., Int J Cancer. 2012 May 15; 130(10):2445-54, each incorporated herein by reference in their entirety. Disclosed herein are compositions comprising poziotinib or a pharmaceutically acceptable salt thereof. Also disclosed are methods for use of poziotinib, in some cases in combination with one or more KRAS inhibitors, for the treatment of KRAS mutant cancer. As used herein, compositions and methods comprising “poziotinib” describe compositions and methods comprising a compound having a structure represented by formula III or a pharmaceutically acceptable salt thereof.

### D. Cancer Immunotherapy

In some embodiments, the methods comprise administration of a cancer immunotherapy as a therapeutic agent. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the cytotoxic T-lymphocyte-associated protein 4 (CTLA-4), also known as CD152. The complete cDNA sequence of human CTLA-4 has the GenBank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the lymphocyte-activation gene 3 (LAG3), also known as CD223 and lymphocyte activating 3. The complete mRNA sequence of human LAG3 has the GenBank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the GenBank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### E. Oncolytic Virus

In some embodiments, a cancer therapy of the present disclosure comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### F. Chemotherapies

In some embodiments, a cancer therapy of the present disclosure comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin, oxaliplatin), substituted ureas (e.g., hydroxyurea), methylhydiazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluorouracil; 5-FU) and floxuridine (fluorodeoxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in certain embodiments of the present disclosure for these cancers as well.

The amount of chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

In some embodiments, a cancer therapy of the present disclosure comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### II. General Pharmaceutical Compositions

In some embodiments, pharmaceutical compositions are administered to a subject. Different aspects may involve administering an effective amount of a composition to a subject. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compounds of the invention may form a solvate which is understood to be a complex of variable stoichiometry formed by a solute (e.g., poziotinib or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. In some embodiments, the solvent is a pharmaceutically acceptable solvent. In some embodiments, the solvent is water.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### III. Kits

Certain aspects of the present disclosure also concern kits containing compositions of the disclosure or compositions to implement methods of the disclosure. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein (e.g., KRAS^{G12C}), which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for one or more biomarkers

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Poziotinib Synergistically Enhances Activity of KRAS G12C Inhibition in Models of KRAS Mutant Cancer

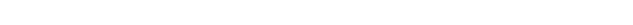

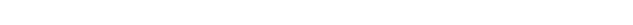

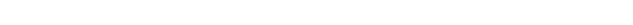

Inhibition of KRAS^{G12C} by AMG 510 (sotorasib) in the KRAS mutant lung cancer cell line H23 resulted in upregulation of pEGFR, pErbB3, and pErbB4 after 4 hours (FIG. 1A). While poziotinib alone had little effect on KRAS mutant cell line viability at concentrations below 10,000 nM, addition of 100 nM poziotinib decreased the IC₅₀ values of the KRAS^{G12C} inhibitors AMG 510 and MRTX849 in KRAS mutant NSCLC cell lines. In H358 cells, IC₅₀ values for AMG 510 and MRTX849 decreased from 8 nM and 28 nM to 0.20 nM and 0.19 nM, respectively. In H1373 cells IC₅₀ values for AMG 510 and MRTX849 decreased from 1,420 nM and 632 nM to 15.0 nM and 22.9 nM, respectively. In H1792 cells, IC₅₀ values for AMG 510 and MRTX849 decreased from >10,000 nM and 1,330 nM to 453 nM and 16.4 nM, respectively. In H2030 cells IC₅₀ values for AMG 510 and MRTX849 decreased from >10,000 nM and 1,360 nM to 472 nM and 3.67 nM, respectively. These results are shown in FIG. 1B (for AMG 510) and FIG. 1C (for MRTX849).

### Example 2—KRAS G12C Inhibitors Increase Phosphorylation of EGFR/HER2

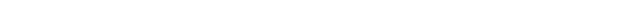

NSCLC cell lines bearing KRAS G12C mutations were treated with sotorasib or adagrasib for 4 hours. pEGFR and pHER2 levels were evaluated by RPPA. KRAS G12C inhibitor treatment resulted in significantly increased levels of pEGFR and pHER2. These results are shown in FIG. 2.

### Example 3—KRAS G12C Inhibitor Treatment Increases Phosphorylation of All EGFR/HER Family Members

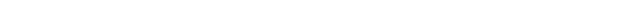

HCC44, H2122, and H358 NSCLC cells (all harboring KRAS G12C mutations) were treated with adagrasib or sotorasib for 72 hours. Phosphorylation of ERBB family members was evaluated by ELISA assay. Results after adagrasib treatment are shown in FIG. 3A. Results after sotorasib treatment are shown in FIG. 3B. In all cell lines, KRAS G12C inhibitor treatment caused an induction of pEGFR, pHER2, pHER3, and pHER4.

### Example 4—Poziotinib has Activity Against HER Family Members Including HER4

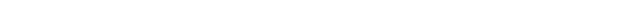

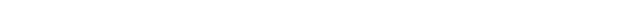

Ba/F3 cells expressing EGFR, EGFR/HER2, HER2/HER3, HER2/HER4, HER3/HER4, and HER4 were generated and tested for sensitivity to poziotinib and afatinib to evaluate the activity of these TKIs against each receptor. Results after Poziotinib treatment are shown in FIG. 4A. Results after afatinib treatment are shown in FIG. 4B. Poziotinib had potent activity against cells expressing HER2/HER3, HER2/HER4, HER3/HER4, and HER4. In contrast. afatinib did not have activity against cells expressing HER3/HER4, and HER4.

### Example 5—Poziotinib is More Selective for HER2-4 Over EGFR Compared to Other TKIs

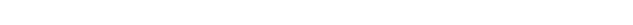

Shown in FIG. 5 are IC₅₀ values for various TKIs, including erlotinib, gefitinib, tucatinib, TAS0728, afatinib, poziotinib, dacomitinib, neratinib, BDTX-189, mobocertinib, lazertinib, and osimertinib. Data was determined based on Ba/F3 cells expressing EGFR, EGFR/HER2, HER2/HER3, HER2/HER4, HER3/HER4, and/or HER4 receptors, and values are IC₅₀ values normalized to Ba/F3 wild-type EGFR IC₅₀.

### Example 6—Exogenous EGF or NRG1 can Promote Resistance to KRAS G12C Inhibitors

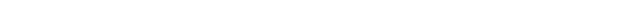

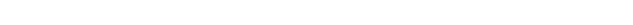

H358 cells (KRAS G12C positive NSCLC) were treated with adagrasib or sotorasib alone or in combination with EGF to activate EGFR or NRG1 to activate other HER family members. FIG. 6A shows resistance of H358 cells to treatment with a combination of adagrasib and exogenous EGF or NRG1. FIG. 6B shows resistance of H358 cells to treatment with a combination of sotorasib and exogenous EGF or NRG1. EGF or NRG1 treatment decreased the sensitivity to adagrasib and sotorasib. These findings support the notion that activation of this pathway may promote resistance to KRAS G12C inhibitors.

### Example 7—The Combination of G12C Inhibitors With Poziotinib is More Synergistic Than EGFR-Specific Inhibitors

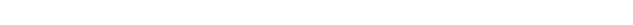

H23, HCC44, H2122, and H1792 cells (NSCLC harboring KRAS G12C mutations) were treated with sotorasib or adagrasib alone or in combination with afatinib or poziotinib. FIG. 7A shows a synergistic effect upon treatment of NSCLC cells harboring KRAS G12C mutations with sotorasib alone or in combination with poziotinib. FIG. 7B shows a synergistic effect upon treatment of NSCLC cells harboring KRAS G12C mutations with adagrasib alone or in combination with poziotinib. Poziotinib, which acts as a pan-HER inhibitor and EGFR inhibitor, yielded a greater synergistic effect than afatinib, which inhibits only EGFR/HER2.

### Example 8—Poziotinib Prevents G12C Inhibitor-Induced Phosphorylation of HER to a Greater Extent Than Afatinib

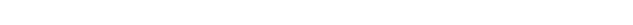

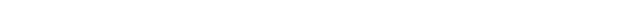

HCC44, H2122, and H358 cells (all NSCLC harboring KRAS G12C mutations) were treated with sotorasib or adagrasib alone or with poziotinib or afatinib. Phosphorylation of ERBB family members pEGFR, pHER2, pHER3, and pHER4 was evaluated by ELISA assay. FIG. 8A shows phosphorylation of ERBB family members in NSCLC cells harboring KRAS G12C mutations treated with sotorasib alone or in combination with afatinib or poziotinib. FIG. 8B shows phosphorylation of ERBB family members in NSCLC cells harboring KRAS G12C mutations treated with adagrasib alone or in combination with afatinib or poziotinib. KRAS G12C inhibitors induced phosphorylation of EGFR and HER family receptors. This effect was inhibited with the addition of poziotinib to a greater extent than with the addition of afatinib.

### Example 9—Low Dose Poziotinib Enhances the In Vivo Activity of KRAS G12C Inhibitors

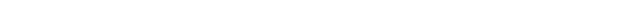

PDX model of KRAS G12C mutant NSCLC was treated with sotorasib alone or in combination with poziotinib (pozi) or afatinib (afat). FIG. 9A shows the effect of sotorasib treatment alone or in combination with poziotinib (pozi) or afatinib (afat) on tumor volume in a PDX model of KRAS G12C mutant NSCLC. FIG. 9B shows the effect of sotorasib treatment alone or in combination with poziotinib (pozi) or afatinib (afat) on progression free survival in a PDX model of KRAS G12C mutant NSCLC. The addition of poziotinib enhanced the anti-tumor activity of sotorasib and extended animal survival.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Canon J, Rex K, Saiki A Y, et al. The clinical KRAS(G12C) inhibitor AMG 510 drives anti-tumour immunity. Nature 2019; 575:217-23.
Hallin J, Engstrom L D, Hargis L, et al. The KRAS(G12C) Inhibitor MRTX849 Provides Insight toward Therapeutic Susceptibility of KRAS-Mutant Cancers in Mouse Models and Patients. Cancer Discov 2020; 10:54-71.
Xue J Y, Zhao Y, Aronowitz J, et al. Rapid non-uniform adaptation to conformation-specific KRAS(G12C) inhibition. Nature 2020; 577:421-5.

What is claimed is:

1. A method of treating a subject for KRAS mutant cancer comprising administering to the subject an effective amount of (a) a KRAS inhibitor and (b) poziotinib.

2. The method of claim 1, wherein the KRAS inhibitor and poziotinib are administered substantially simultaneously.

3. The method of claim 1, wherein the KRAS inhibitor and poziotinib are administered sequentially.

4. The method of claim 1, wherein the KRAS inhibitor is administered prior to administering poziotinib.

5. The method of claim 1, wherein the KRAS inhibitor is administered subsequent to administering poziotinib.

6. The method of any of claims 1-5, wherein the KRAS inhibitor is a KRAS^{G12C} inhibitor.

7. The method of claim 6, wherein the KRAS^{G12C} inhibitor is sotorasib (AMG 510).

8. The method of claim 6, wherein the KRAS^{G12C} inhibitor is adagrasib (MRTX849).

9. The method of any of claims 1-5, further comprising detecting a KRAS^{G12C} mutation in the subject.

10. The method of any of claims 1-9, wherein the subject was previously treated with a cancer therapy.

11. The method of claim 10, wherein the cancer therapy comprised chemotherapy.

12. The method of claim 10, wherein the cancer therapy comprised the KRAS inhibitor.

13. The method of any of claims 10-12, wherein the subject was determined to be resistant to the cancer therapy.

14. The method of any of claims 1-9, wherein the subject was not previously treated with the KRAS inhibitor.

15. The method of any of claims 1-14, wherein the KRAS mutant cancer is KRAS mutant non-small cell lung cancer.

16. The method of any of claims 1-14, wherein the KRAS mutant cancer is KRAS mutant colorectal cancer.

17. The method of any of claims 1-14, wherein the KRAS mutant cancer is KRAS mutant pancreatic cancer.

18. The method of any of claims 1-17, wherein the poziotinib is administered at a dose of between 0.1 mg and 50 mg.

19. The method of claim 18, wherein the poziotinib is administered at a dose of between 1 mg and 25 mg.

20. The method of claim 18, wherein the poziotinib is administered at a dose of between 1 mg and 5 mg.

21. The method of any of claims 1-19, wherein the poziotinib is administered orally.

22. The method of any of claims 1-21, wherein the KRAS inhibitor and poziotinib are administered once per day for multiple days.

23. The method of any of claims 1-21, wherein the KRAS inhibitor and poziotinib are administered twice per day for multiple days.

24. The method of any of claims 1-23, further comprising administering to the subject an additional cancer therapy.

25. The method of claim 24, wherein the additional cancer therapy comprises chemotherapy, radiotherapy, immunotherapy, or a combination thereof.

26. A pharmaceutical composition comprising:
(a) a KRAS inhibitor;
(b) poziotinib; and
(c) a pharmaceutically acceptable excipient.

27. The pharmaceutical composition of claim 26, wherein the KRAS inhibitor is a KRAS^{G12C} inhibitor.

28. The pharmaceutical composition of claim 27, wherein the KRAS^{G12C} inhibitor is sotorasib (AMG 510).

29. The pharmaceutical composition of claim 27, wherein the KRAS^{G12C} inhibitor is adagrasib (MRTX849).

30. The pharmaceutical composition of any of claims 26-29, wherein the poziotinib is at a dose of between 0.1 mg and 50 mg.

31. The pharmaceutical composition of claim 30, wherein the poziotinib is at a dose of between 1 mg and 25 mg.

32. The pharmaceutical composition of claim 30, wherein the poziotinib is at a dose of between 1 mg and 5 mg.

33. A method of treating a subject for KRAS^{G12C} non-small cell lung cancer, the method comprising administering to the subject an effective amount of:
(a) a KRAS^{G12C} inhibitor; and
(b) poziotinib.

34. The method of claim 33, wherein the KRAS^{G12C} inhibitor and poziotinib are administered substantially simultaneously.

35. The method of claim 33, wherein the KRAS^{G12C} inhibitor and poziotinib are administered sequentially.

36. The method of claim 33, wherein the KRAS^{G12C} inhibitor is administered prior to administering poziotinib.

37. The method of claim 33, wherein the KRAS^{G12C} inhibitor is administered subsequent to administering poziotinib.

38. The method of any of claims 33-37, wherein the KRAS^{G12C} inhibitor is sotorasib.

39. The method of any of claims 33-37, wherein the KRAS^{G12C} inhibitor is adagrasib.

40. The method of any of claims 33-39, wherein the subject was previously treated with a cancer therapy.

41. The method of claim 40, wherein the cancer therapy comprised chemotherapy.

42. The method of claim 40, wherein the cancer therapy comprised the KRAS^{G12C} inhibitor.

43. The method of any of claims 40-42, wherein the subject was determined to be resistant to the cancer therapy.

44. The method of any of claims 33-37, wherein the subject was not previously treated with sotorasib or adagrasib.

45. The method of any of claims 33-44, wherein the poziotinib is administered at a dose of between 0.1 mg and 50 mg.

46. The method of claim 45, wherein the poziotinib is administered at a dose of between 1 mg and 25 mg.

47. The method of claim 45, wherein the poziotinib is administered at a dose of between 1 mg and 5 mg.

48. The method of any of claims 33-46, wherein the poziotinib is administered orally.

49. The method of any of claims 33-48, wherein the KRAS^{G12C} inhibitor and poziotinib are administered once per day for multiple days.

50. The method of any of claims 33-48, wherein the KRAS^{G12C} inhibitor and poziotinib are administered twice per day for multiple days.

51. The method of any of claims 33-50, further comprising administering to the subject an additional cancer therapy.

52. The method of claim 51, wherein the additional cancer therapy comprises chemotherapy, radiotherapy, immunotherapy, or a combination thereof.

53. A method of treating a subject for KRAS^{G12C} non-small cell lung cancer, the method comprising administering to the subject, twice daily for multiple days, an effective amount of:
(a) a KRAS^{G12C} inhibitor; and
(b) poziotinib at a dose of between 1 mg and 5 mg.
